SEQUENCE LISTING (4 sequences)

SEQ ID NO: 1 - protein, 352 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 3 - protein, 1130 aa, Homo sapiens [sequence omitted]

SEQ ID NO: 4 - protein, 1182 aa, Homo sapiens
MGQQVGRVGEAPGLQQPQPRGIRGSSAARPSGRRRDPAGRTTETGFNIFTQHDHFASCVEDGFEGDKTGGSSPEALHRPYGCDVEPQALNEAIRWSSKENLLGATESDPNLFVALYDFVASGDNTLSITKGEKLRVLGYNQNGEWSEVRSKNGQGWVPSNYITPVNSLEKHSWYHGPVSRSAAEYLLSSLINGSFLVRESESSPGQLSISLRYEGRVYHYRINTTADGKVYVTAESRFSTLAELVHHHSTVADGLVTTLHYPAPKCNKPTVYGVSPIHDKWEMERTDITMKHKLGGGQYGEVYVGVWKKYSLTVAVKTLKEDTMEVEEFLKEAAVMKEIKHPNLVQLLGVCTLEPPFYIVTEYMPYGNLLDYLRECNREEVTAVVLLYMATQISSAMEYLEKKNFIHRDLAARNCLVGENHVVKVADFGLSRLMTGDTYTAHAGAKFPIKWTAPESLAYNTFSIKSDVWAFGVLLWEIATYGMSPYPGIDLSQVYDLLEKGYRMEQPEGCPPKVYELMRACWKWSPADRPSFAETHQAFETMFHDSSISEEVAEELGRAASSSSVVPYLPRLPILPSKTRTLKKQVENKENIEGAQDATENSASSLAPGFIRGAQASSGSPALPRKQRDKSPSSLLEDAKETCFTRDRKGGFFSSFMKKRNAPTPPKRSSSFREMENQPHKKYELTGNFSSVASLQHADGFSFTPAQQEANLVPPKCYGGSFAQRNLCNDDGGGGGGSGTAGGGWSGITGFFTPRLIKKTLGLRAGKPTASDDTSKPFPRSNSTSSMSSGLPEQDRMAMTLPRNCQRSKLQLERTVSTSSQPEENVDRANDMLPKKSEESAAPSRERPKAKLLPRGATALPLRTPSGDLAITEKDPPGVGVAGVAAAPKGKEKNGGARLGMAGVPEDGEQPGWPSPAKAAPVLPTTHNHKVPVLISPTLKHTPADVQLIGTDSQGNKFKLLSEHQVTSSGDKDRPRRVKPKCAPPPPPVMRLLQHPSICSDPTEEPTALTAGQSTSETQEGGKKAALGAVPISGKAGRPVMPPPQVPLPTSSISPAKMANGTAGTKVALRKTKQAAEKISADKISKEALLECADLLSSALTEPVPNSQLVDTGHQLLDYCSGYVDCIPQTRNKFAFREAVSKLELSLQELQVSSAAAGVPGTNPVLNNLLSCVQEISDVVQR

(19) United States
(12) Patent Application Publication
(10) Pub. No.: US 2007/0134724 A1
(43) Pub. Date: Jun. 14, 2007

(54) Phosphorylation of tau by abl

(76) Inventors: Peter Davies, Rye, NY (US); Christopher Conrad, Mount Vernon, NY (US)

Correspondence Address: AMSTER, ROTHSTEIN & EBENSTEIN LLP, 90 PARK AVENUE, NEW YORK, NY, 10016, US

(21) Appl. No.: 11/498,339

(22) Filed: Aug. 3, 2006

Related U.S. Application Data
(60) Provisional application No. 60/705,585, filed on Aug. 4, 2005.

(51) Int. Cl. G01N 33/53; G01N 33/567; C07K 16/28

(52) U.S. Cl. 435/7.1; 435/7.2; 530/388.22

(57) ABSTRACT

Methods of diagnosing a tauopathy and predicting whether a subject will develop a tauopathy are provided. Also provided are antibody preparations that specifically bind to tau phosphorylated at tyr394 and/or tyr310. Methods of inhibiting tau phosphorylation in a cell and methods of treating a subject having a tauopathy are additionally provided. Methods of treating a subject at risk for a tauopathy are also provided. Additionally, non-human mammals comprising a transgene encoding an abl tyrosine kinase are provided. Also provided are methods of evaluating whether a compound inhibits development of a tauopathy.

### CROSS-REFERENCE TO RELATED APPLICATION

This application claims the benefit of U.S. Provisional Application No. 60/705,585, filed Aug. 4, 2005.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of NIMH38623 awarded by The National Institutes for Mental Health.

### BACKGROUND OF THE INVENTION

(1) Field of the Invention

The present invention generally relates to diagnosis and treatment of tauopathies. More specifically, the invention relates to phosphorylation of tau by abl tyrosine kinases and diagnoses and treatments of tauopathies, including Alzheimer's disease, directed to that phosphorylation.

(2) Description of the Related Art

References Cited:

Andreasen, N. (2003) Brain Aging 3:7-14.
Binder, L. I. et al. (2005) Biochim. Biophys. Acta 1739:216-223.
Brant, R. (1996) Front. Biosci. 1:d118-130.
Conrad, C. G. (2003) Saitohin: A Polymorphic Gene in the tau Locus. Ph.D. Thesis, Albert Einstein College of Medicine of Yeshiva University.
Dan, S. et al. (1998) Cell Death Different. 5:710-715.
Deininger, M. W. N. et al. (1997) Blood 90:3691-3698.
Derkinderen, P. et al. (2005) J. Neurosci. 25:6548-6593.
Ferrer, I. et al. (2001) Brain Pathol. 11:144-158.
Forman, M. S. (2002) Am. J. Pathol. 160:1725-1731.
Friedhoff, P. et al. (2000) Biochim. Biophys. Acta 1502:122-132.
Hampel, H. et al. (2004) Arch. Gen. Psychiatry 61:95-102.
Johnson, G. V. W. and Stoothoff, W. H. (2004) J. Cell Sci. 117:5721-5729.
Lee, G. et al. (2004) J. Neurosci. 24:2304-2312.
Lee, G. et al. (1998) J. Cell Sci. 111:3167-2177.
Marsh, H. N. (1998) Neural Notes 111: 17-20.
Netzer, W. J. et al. (2003) Proc. Natl. Acad. Sci. USA 100:12444-12449.
O'Hare, T. et al. (2004) Blood 104:2532-2539.
Shaul, Y. (2000) Cell Death Differentiation 7:10-16.
Stoothoff, W. H. and Johnson, G. V. W. (2005) Biochim. Biophys. Acta 1739:280-297.
Trojanowski, J. Q. and Lee, V. M.-Y. (1995) FASEB 9:1-570-1576.
Uboga, N. V. and Prise, J. L. (2000) Neurobiol. Aging 21:1-10.
Warmuth, M. et al. (2003) Blood 101:664-672.
Weaver, C. L. et al. (2000) Neuobiol. Aging 21:719-727.
Young, M. A. et al. (2006) Cancer Res. 66:1007-1014.

One of the hallmarks of Alzheimer's disease is neurofibrillary tangles (NFTs), which comprise aggregates of filamentous polymers of tau, a microtubule-associated protein (Binder et al., 2005; Friedhoff et al., 2000). Hyperphosphorylation of tau is associated with NFTs and is involved in other diseases involving abnormal tau (tauopathies)(Stoothoff and Johnson, 2005; Lee et al., 1998; Lee et al., 2004; Andreasen, 2003; Trojanowski and Lee, 1995; Ferrer et al., 2001). This phosphorylation has been identified at several serine and threonine residues, in addition to the tyrosine at residue 18 (tyr18) (Stoothoff and Johnson, 2005; Lee et al., 2004). Tyr18 is believed to be phosphorylated by fyn, the src family tyrosine kinase (Lee et al., 2004).

Although the tyrosine kinase abl has not been previously evaluated for its effect of tau, the abl inhibitor Gleevec (imatinib mesylate, ST1571) inhibits the production of Aβ (another protein associated with Alzheimer's disease) in neurons and guinea pig brains (Netzer et al., 2003). However, Gleevec has not been evaluated for its effect on tau phosphorylation or in Alzheimer's disease or an animal model thereof.

Further evaluation of tyrosine phosphorylation of tau and its effect on tauopathies including Alzheimer's disease is needed. The present invention addresses that need.

### SUMMARY OF THE INVENTION

Accordingly, the present invention is based on the discovery that abl tyrosine kinases phosphorylate tau and are present in neurofibrillary tangles in Alzheimer's disease patients.

Thus, in some embodiments, the invention is directed to methods of diagnosing a tauopathy in a subject. The methods comprise determining whether the subject has tyrosine phosphorylation of tau at tyr394 or tyr310, where tyrosine phosphorylation of tau at tyr394 or tyr310 in the subject indicates that the subject has a tauopathy.

In other embodiments, the invention is directed to methods of predicting whether a subject will develop a tauopathy. The methods comprise determining whether the subject has tau phosphorylated at tyr394 or tyr310, where the presence in the subject of tau phosphorylated at tyr394 or tyr310 indicates the subject will develop the tauopathy.

The invention is also directed to antibody preparations that specifically bind to tau phosphorylated at tyr394 and/or tyr310.

In further embodiments, the invention is directed to methods of inhibiting tau phosphorylation in a cell. The methods comprise combining the cell with an inhibitor of an abl tyrosine kinase in a manner sufficient to inhibit tau phosphorylation in the cell.

Additionally, the invention is directed to methods of treating a subject having a tauopathy. The methods comprise administering an inhibitor of an abl tyrosine kinase to the subject in a manner sufficient to inhibit tau tyrosine phosphorylation in a neuron in the subject.

The invention is also directed to methods of treating a subject at risk for a tauopathy. The methods comprise administering an inhibitor of an abl tyrosine kinase to the subject in a manner sufficient to inhibit tau tyrosine phosphorylation in a neuron in the subject.

The invention is additionally directed to non-human mammals comprising a transgene encoding an abl tyrosine kinase such that the abl tyrosine kinase is expressed in a neuron of the mammal.

In additional embodiments, the invention is directed to methods of evaluating whether a compound inhibits development of a tauopathy. The methods comprise combining the compound with an abl tyrosine kinase and determining whether the compound inhibits the abl tyrosine kinase. In these embodiments, a compound that inhibits they abl tyrosine kinase inhibits development of the tauopathy.

### BRIEF DESCRIPTION OF THE DRAWINGS

FIG. 1 is micrographs of Alzheimer's disease (AD) brain tissue sections stained with abl antibodies. Panels A-D are sections stained with the anti-abl antibody K12 (Santa Cruz Biotechnology). Panel A shows an Alzheimer's disease brain section stained with a K12 antibody preparation that has been cross-absorbed with the abl protein. Panels B and C are images from an early AD case, showing the presence of abl associated with both plaques and tangles. Panel D is a close up view of a tangle stained by abl antibodies in early AD. Panels E-H are pictures anti-abl antibody AB-1 (Oncogene Biosciences). Panel E shows a section stained with antibody AB-1 that has been cross-absorbed with abl protein. Panel F are images from a moderate case of AD. Panel G is a higher power image from the moderate case of AD. Panel H is an even higher power image from the moderate case of AD.

FIG. 2 is micrographs of AD brain tissue sections stained with a monoclonal antibody that recognizes tau when phosphorylated at either tyrosine 18 or tyrosine 29. This antibody does not recognize anything in the normal adult human brain. Panels A-D are images taken from an early case of Alzheimer's disease. Panel A shows a neuron in the brain of the early AD patient. Panel B shows two cells, one with a tangle (bottom left) and one which is apparently an early tangle (upper right). Panel C shows that cells with tangles stained throughout the cell and the cell processes are clearly visible. Panel D shows the neuronal processes that surround amyloid deposits. Panel E shows tyrosine phosphorylation in a more advanced AD case. Panel F shows another view of the advanced AD case.

FIG. 3 is photographs of blots of electrophoresed SDS-PAGE gels of tau from cells transfected with abl (Abl), fyn (Fyn), or nothing (NT). The blots were then treated with a mouse monoclonal antibody that binds to tau, then treated with horseradish peroxidase (HRP)-labeled goat anti-mouse IgG, then developed with an HRP substrate and photographed. Antibody CP27 binds to all forms of tau, whether phosphorylated or not; antibody 9G3 binds only to tau that is phosphorylated at tyrosines 18 and 29; antibody 4G10 binds to tau that is phosphorylated at any tyrosine.

FIG. 4 is photographs of blots stained as in FIG. 3 of five mutant tau proteins that were phosphorylated by abl. The five mutants each had one tyrosine substituted with phenylalanine—at residues 18 (Y18F), 29 (Y29F), 197 (Y197F), 310 (Y310F), and 394 (Y394F). After electrophoresis and blotting, the blots were stained with the antibodies described in the brief description of FIG. 3 above.

FIG. 5 is graphs and photographs of western blots further establishing that abl2 phosphorylates tau at tyrosine-394 (Y394) and tyrosine 310 (Y310). Panel A shows western blots of cell lysates of Y-to-F mutants showing Y394 as a major phosphorylation site, with lower levels of phosphorylation at Y197 and Y310. Panel B shows western blots further establishing that Y394 is the predominant site of abl2 phosphorylation in short (3R) and long (4R) isoforms of tau. Panels C and D are graphs of sandwich ELISA results that confirm the western blotting results indicating Y394 as the major phosphorylation site for both the longest (C) and shortest (D) isoforms of tau.

FIG. 6 is a graph and photographs of western blots further establishing that Y310 is phosphorylated by both abl1 (Abl) and abl2 (Arg), using a monoclonal antibody, YP21, that specifically recognizes tau phosphorylated at Y310. Panel A is a graph of ELISA results showing the specificity of YP21 for phospho-Y310. Panels B and C show western blots demonstrating phosphorylation of tau at Y310, and a complete loss of YP21 immunoreactive when Y310 is mutated.

FIG. 7 is graphs and photographs of western blots establishing that abl1 and abl2 are each capable of mediating tyrosine phosphorylation of tau independently. Panels A and B show western blots establishing that phosphorylation of Y310 by wild-type abl1 (A) or abl2 (B), but not Gleevec-resistant abl1 or abl2, is inhibited by imatinib mesylate (Gleevec). Panels C and D further support the western blot data with sandwich ELISA.

FIG. 8 is graphs of ELISA results showing that the YP3 and YP4 antibodies are specific for tau having the dual phosphorylation of phosphotyrosine 394/phosphoserine 396.

FIG. 9 is micrographs showing staining of brain tissue with the YP3 or YP4 antibodies, showing the presence of large amounts of phosphotyrosine 394/phosphoserine 396 tau in the Alzheimer's brain, in association with plaques, tangles and abnormal neurites. Panel A shows staining of Alzheimer's tissues with YP3; Panel B shows staining of Alzheimer's tissues with YP4; Panel C shows staining of normal brain with YP3.

### DETAILED DESCRIPTION OF THE INVENTION

The inventors have discovered that abl tyrosine kinases phosphorylate tau and are present in neurofibrillary tangles in Alzheimer's disease patients. See Examples. Based in part on this discovery and the realization of the connection between abl and cell cycle activation (as discussed in the Example) tying together the association of abl in tangles and the etiology of Alzheimer's, the inventors have developed methods and compositions for diagnosis and treatment of tauopathies, including Alzheimer's disease.

Thus, the invention is directed to methods of diagnosing a tauopathy in a subject. The methods comprise determining whether the subject has tyrosine phosphorylation of tau at tyr394 or tyr310, where tyrosine phosphorylation of tau at tyr394 or tyr310 in the subject indicates that the subject has a tauopathy.

As established in the Example 1, abl is present in brains of Alzheimer's patients in association with plaques and tangles, and abl phosphorylates tyr394 of tau. Since the amino acids surrounding tyr310 in tau are very similar to the amino acids surrounding tyr394 (see SEQ ID NO:1), the skilled artisan would expect abl to phosphorylate tyr310. This expectation is confirmed in experiments described in Example 2.

As used herein, tau is a microtubule-associated protein translated from the human chromosomal sequence of GenBank Accession No. AH005895, or naturally occurring mammalian variants thereof. As is known, due to alternative splicing, there are several isoforms of the tau protein. Six human brain isoforms of tau are currently known (Brandt, 1996). The shortest known isoform, tau352, is provided herein as SEQ ID NO:1; the longest known isoform, tau441, has the sequence of SEQ ID NO:2. By convention, the amino acids are named according to the numbering of the longest isoform (SEQ ID NO:2-441 amino acids). That isoform has tyrosines at amino acids 18, 29, 197, 310, and 394. However, as used herein, those five tyrosines, which are present in all isoforms, have the same amino acid designations with all of the isoforms. Thus, tyrosine-394 (tyr394 or Y394) has that designation with the analogous tyrosine residue for any of the isoforms, even those isoforms having less than 394 amino acids. Similarly, tyrosine-310 (tyr310 or Y310) has that designation with the analogous tyrosine residue for any of the isoforms.

These methods preferably further comprise determining whether the subject has phosphorylation of tau at a second or more amino acid residues. The second amino acid residue can be a tyrosine or any other tau amino acid residue now known or later discovered to be subject to phosphorylation in a tauopathy. See, e.g., Johnson and Stoothoff, 2004.

Serine 396 of tau is always phosphorylated in Alzheimer's disease (Uboga and Price, 2000; Weaver et al., 2000). Therefore abl1 or abl2 activity should produce the dual phosphorylated site phosphotyrosine 394/phosphoserine 396. The presence of this epitope was confirmed in the experiments described in Example 2. The present methods thus preferably comprise determining whether the subject has phosphorylation of tau at tyr394 and ser396. The phosphorylation of tau at tyr394 and ser396 is preferably determined using an antibody that binds specifically to tau phosphorylated at tyr394 and ser396. Preferred examples of such antibodies are YP3 or YP4.

Where phosphorylation of a second amino acid residue is determined, the second amino acid residue can also preferably be a tyrosine. In these aspects, the method further comprises determining whether the subject has phosphorylation of tau at tyr394 and tyr310.

These methods are useful for the diagnosis of any tauopathy associated with tau having phosphorylated tyrosines. Tauopathies included in these embodiments are frontotemportal dementia, progressive supernuclear palsy, Pick's disease, corticobasal degeneration, Parkinson's disease and Lewy body dementia (see, e.g., Ferrer et al., 2001, Forman et al., 2002; Marsh, 1998; Johnson and Stoothoff, 2004). Preferably, the tauopathy is Alzheimer's disease.

Preferably, the tyrosine phosphorylation of tau at tyr394 or tyr310 is determined in a bodily fluid of the subject, preferably peripheral blood or cerebrospinal fluid.

Phosphorylation of tau at tyr394 or tyr310 can be determined by any known method. In preferred embodiments, tyrosine phosphorylation of tau at tyr394 or tyr310 is determined using an antibody specific for tau phosphorylated at tyr394 or tyr310, for example by western blot (see, e.g., Example and Hampel et al., 2004).

The invention is also directed to methods of predicting whether a subject will develop a tauopathy. The methods comprise determining whether the subject has tau phosphorylated at tyr394 or tyr310, where the presence in the subject of tau phosphorylated at tyr394 or tyr310 indicates the subject will develop the tauopathy. In some embodiments, phosphorylation at tyr394 is determined; in others, phosphorylation at tyr310 is determined.

As with the previously described methods, these methods preferably further comprise determining whether the subject has phosphorylation of tau at a second or more amino acid residues. The second amino acid residue can be a tyrosine or any other tau amino acid residue now known or later discovered to be subject to phosphorylation. Preferably, these methods further comprise determining whether the subject has phosphorylation of tau at tyr394 and ser396, most preferably using an antibody that binds specifically to tau phosphorylated at tyr394 and ser396, e.g., antibody YP3 or YP4.

Where phosphorylation of a second amino acid residue is determined, the second amino acid residue can also preferably be a tyrosine. In these aspects, the method further comprises determining whether the subject has phosphorylation of tau at tyr394 and tyr310.

As with the methods for diagnosing a tauopathy described above, these methods are useful for the diagnosis of any tauopathy associated with tau having phosphorylated tyrosines. Tauopathies included in these embodiments are frontotemportal dementia, progressive supemuclear palsy, Pick's disease, corticobasal degeneration, Parkinson's disease, and Lewy body dementia. Preferably, the tauopathy is Alzheimer's disease.

Preferably in these methods, the tyrosine phosphorylation of tau at tyr394 or tyr310 is determined in a bodily fluid of the subject, preferably peripheral blood or cerebrospinal fluid.

Phosphorylation of tau at tyr394 or tyr310 in these methods can be determined by any known method. In preferred embodiments, tyrosine phosphorylation of tau at tyr394 or tyr310 is determined using an antibody specific for tau phosphorylated at tyr394 or tyr310, for example by western blot.

The invention is also directed to antibody preparations that specifically bind to tau phosphorylated at tyr394 and/or tyr310. Included herewith are antibodies that are specific for tau phosphorylated at tyr394, antibodies that are specific for tau phosphorylated at tyr310, and antibodies that are specific for tau phosphorylated at either tyr394, tyr310 or both.

The antibody of these preparations preferably binds to the tyr394 or tyr310 when another amino acid residue proximal to the tyr394 or tyr310 is phosphorylated. In these embodiments, the additional amino acid residue is proximal to the tyr394 or tyr310 three dimensionally, and need not necessarily be proximal to the tyr394 or tyr310 in the primary sequence. An example of a proximal amino acid is ser396. Thus, a preferred antibody preparation specifically binds to tau phosphorylated at tyr394 and ser396. Examples of such preparations comprise antibody YP3 and/or YP4.

These preparations can be monoclonal antibodies, polyclonal antibodies, single chain antibodies, an antibody fragment comprising an antibody binding site (e.g., an Fab or an Fab2 fragment) or antibodies or antibody fragments produced using genetic engineering methods such as (but not limited to) antibodies produced using phage display technology. Also included herewith are heterologous proteins that include antibody binding sites.

In further embodiments, the invention is directed to methods of inhibiting tau phosphorylation in a cell. The methods comprise combining the cell with an inhibitor of an abl tyrosine kinase in a manner sufficient to inhibit tau phosphorylation in the cell.

As used herein, an abl tyrosine kinase is a mammalian protein having the amino acid sequence of SEQ ID NO:3 or SEQ ID NO:4, or mammalian variants thereof that have tyrosine kinase activity. Those sequences are of abl1 (also known as c-abl)(Shaul et al., 2000) and abl2 (also known as arg).

In some embodiments of these methods, the abl tyrosine kinase is abl1; in other embodiments the abl tyrosine kinase is abl2. The tau phosphorylation in these embodiments can be at tyr394 or tyr310, or both amino acids.

These methods can be used to inhibit tau phosphorylation in any cell, including any eukaryotic, prokaryotic or archaeal cells that have been transformed with tau. In preferred embodiments, the cell is a mammalian neuron. The neuron can be in culture or in a living mammal. Where the mammal is in a human, the human preferably is at risk for, or has a tauopathy, such as frontotemportal dementia, progressive supemuclear palsy, Pick's disease, corticobasal degeneration, Parkinson's disease, Lewy body dementia, or preferably Alzheimer's disease.

In these methods, the inhibitor is preferably selective for non-receptor tyrosine kinases, most preferably abl tyrosine kinases. In additional preferred embodiments, the inhibitor is a small organic molecule. Several such inhibitors are known. Preferred examples include ST1571 (Gleevec, imatinib mesylate), CGP 57148, AG1112, AP23464, CGP76030, or PP1 (Netzer et al., 2003; Dan et al., 1998; Deininger et al., 1997; O'Hare et al., 2004; Warmuth et al., 2003).

The inhibitor can alternatively be a macromolecule that specifically binds to the abl tyrosine kinase, for example an antibody (including antibody fragments or heterologous proteins comprising an antibody binding site, as previously discussed) or an aptamer.

Aptamers are single stranded oligonucleotides or oligonucleotide analogs that bind to a particular target molecule, such as a protein or a small molecule (e.g., a steroid or a drug, etc.). Thus, aptamers are the oligonucleotide analogy to antibodies. However, aptamers are smaller than antibodies, generally in the range of 50-100 nt. Their binding is highly dependent on the secondary structure formed by the aptamer oligonucleotide. Both RNA and single stranded DNA (or analog), aptamers are known. See, e.g., U.S. Pat. Nos. 5,773,598; 5,496,938; 5,580,737; 5,654,151; 5,726,017; 5,786,462; 5,503,978; 6,028,186; 6,110,900; 6,124,449; 6,127,119; 6,140,490; 6,147,204; 6,168,778; and 6,171,795.

Aptamers that bind to virtually any particular target can be selected by using an iterative process called SELEX, which stands for Systematic Evolution of Ligands by EXponential enrichment. Several variations of SELEX have been developed which improve the process and allow its use under particular circumstances. See, e.g., U.S. Pat. Nos. 5,472,841; 5,503,978; 5,567,588; 5,582,981; 5,637,459; 5,683,867; 5,705,337; 5,712,375; and 6,083,696. Methods for expressing aptamers from vectors have recently been developed (PCT Publication No. WO 03/102146).

These inhibitors can also be vectors comprising a nucleic acid sequence that is homologous to a portion of a polynucleotide in the cell encoding the abl tyrosine kinase. Nonlimiting examples of such nucleic acid sequences are, or encode, microRNAs, antisense RNAs, and ribozymes that inhibit transcription or translation of the polynucleotide encoding the abl tyrosine kinase. Such inhibitors could be produced by the skilled artisan without undue experimentation.

Most preferably, the inhibitor is formulated in a pharmaceutical composition that enhances the ability of the compound to cross the blood-brain barrier of a mammal. By “pharmaceutically acceptable” it is meant a material that (i) is compatible with the other ingredients of the composition without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers include, without limitation, any of the standard pharmaceutical carriers such as phosphate buffered saline solutions, water, emulsions such as oil/water emulsions, microemulsions, and the like.

The above-described compounds can be formulated without undue experimentation for administration to a mammal, including humans, as appropriate for the particular application. Additionally, proper dosages of the compositions can be determined without undue experimentation using standard dose-response protocols.

Accordingly, the compositions designed for oral, lingual, sublingual, buccal and intrabuccal administration can be made without undue experimentation by means well known in the art, for example with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums and the like.

Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, cornstarch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and nontoxic in the amounts used.

The compounds can easily be administered parenterally such as for example, by intravenous, intramuscular, intrathecal or subcutaneous injection. Parenteral administration can be accomplished by incorporating the compounds into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the compound, in a pharmaceutical composition, into the rectum or large intestine. This can be accomplished using suppositories or enemas. Suppository formulations can easily be made by methods known in the art. For example, suppository formulations can be prepared by heating glycerin to about 120° C., dissolving the composition in the glycerin, mixing the heated glycerin after which purified water may be added, and pouring the hot mixture into a suppository mold.

Transdermal administration includes percutaneous absorption of the composition through the skin. Transdermal formulations include patches (such as the well-known nicotine patch), ointments, creams, gels, salves and the like.

The present invention includes nasally administering to the mammal a therapeutically effective amount of the compound. As used herein, nasally administering or nasal administration includes administering the compound to the mucous membranes of the nasal passage or nasal cavity of the patient. As used herein, pharmaceutical compositions for nasal administration of the compound include therapeutically effective amounts of the compound prepared by well-known methods to be administered, for example, as a nasal spray, nasal drop, suspension, gel, ointment, cream or powder. Administration of the compound may also take place using a nasal tampon or nasal sponge.

Where the compound is administered peripherally such that it must cross the blood-brain barrier, the compound is preferably formulated in a pharmaceutical composition that enhances the ability of the compound to cross the blood-brain barrier of the mammal. Such formulations are known in the art and include lipophilic compounds to promote absorption. Uptake of non-lipophilic compounds can be enhanced by combination with a lipophilic substance. Lipophilic substances that can enhance delivery of the compound across the nasal mucus include but are not limited to fatty acids (e.g., palmitic acid), gangliosides (e.g., GM-1), phospholipids (e.g., phosphatidylserine), and emulsifiers (e.g., polysorbate 80), bile salts such as sodium deoxycholate, and detergent-like substances including, for example, polysorbate 80 such as Tween™, octoxynol such as Triton™ X-100, and sodium tauro-24,25-dihydrofusidate (STDHF). See Lee et al., Biopharm., April 1988 issue:3037.

The compound can be combined with micelles comprised of lipophilic substances. Such micelles can modify the permeability of the nasal membrane to enhance absorption of the compound. Suitable lipophilic micelles include without limitation gangliosides (e.g., GM-1 ganglioside), and phospholipids (e.g., phosphatidylserine). Bile salts and their derivatives and detergent-like substances can also be included in the micelle formulation. The compound can be combined with one or several types of micelles, and can further be contained within the micelles or associated with their surface.

The compound can also be conjugated or coupled to agents that increase the lipophilicity of the compound (thus increasing blood brain barrier penetration) or are subject to active transport. Such agents are known in the art.

Alternatively, the compound can be combined with liposomes (lipid vesicles) to enhance absorption. The compound can be contained or dissolved within the liposome and/or associated with its surface. Suitable liposomes include phospholipids (e.g., phosphatidylserine) and/or gangliosides (e.g., GM-1). For methods to make phospholipid vesicles, see for example, U.S. Pat. No. 4,921,706 to Roberts et al., and U.S. Pat. No. 4,895,452 to Yiournas et al. Bile salts and their derivatives and detergent-like substances can also be included in the liposome formulation.

The present invention is also directed to methods of treating a subject having a tauopathy. The methods comprise administering an inhibitor of an abl tyrosine kinase to the subject in a manner sufficient to inhibit tau tyrosine phosphorylation in a neuron in the subject. Analogous to the methods described above, the abl tyrosine kinase can be abl1 or abl2. Additionally, the tauopathy can be any tauopathy involving tyrosine phosphorylation, for example frontotemportal dementia, progressive supernuclear palsy, Pick's disease, corticobasal degeneration, Parkinson's disease, or Lewy body dementia. Preferably, the tauopathy is Alzheimer's disease.

In these methods, the inhibitor is preferably selective for non-receptor tyrosine kinases, most preferably abl tyrosine kinases. In additional preferred embodiments, the inhibitor is a small organic molecule. Several such inhibitors are known. Preferred examples include ST1571, CGP57148, AG1112, AP23464 or PP1.

The inhibitor can alternatively be a macromolecule that specifically binds to the abl tyrosine kinase, for example an antibody or an aptamer.

The inhibitor can also be a vector comprising a nucleic acid sequence that is homologous to a portion of a polynucleotide in the cell encoding the abl tyrosine kinase. Examples of such nucleic acid sequences are, or encode, microRNAs, antisense RNAs, and ribozymes that inhibit transcription or translation of the polynucleotide encoding the abl tyrosine kinase.

Preferably, the inhibitor is formulated in a pharmaceutical composition that enhances the ability of the compound to cross the blood-brain barrier of a mammal. The inhibitor can also be administered directly to the brain of the mammal.

The invention is also directed to methods of treating a subject at risk for a tauopathy. The methods comprise administering an inhibitor of an abl tyrosine kinase to the subject in a manner sufficient to inhibit tau tyrosine phosphorylation in a neuron in the subject. Analogous to the methods described above, the abl tyrosine kinase can be abl1 or abl2. Additionally, the tauopathy can be any tauopathy involving tyrosine phosphorylation, for example frontotemportal dementia, progressive supernuclear palsy, Pick's disease, corticobasal degeneration, Parkinson's disease, and Lewy body dementia. Preferably, the tauopathy is Alzheimer's disease.

In these methods, the inhibitor is preferably selective for non-receptor tyrosine kinases, most preferably abl tyrosine kinases. In additional preferred embodiments, the inhibitor is a small organic molecule. Several such inhibitors are known. Preferred examples include ST1571, CGP57148, AG1112, AP23464 or PP1.

The inhibitor can alternatively be a macromolecule that specifically binds to the abl tyrosine kinase, for example an antibody or an aptamer.

The inhibitor can also be a vector comprising a nucleic acid sequence that is homologous to a portion of a polynucleotide in the cell encoding the abl tyrosine kinase. Examples of such nucleic acid sequences are, or encode, microRNAs, antisense RNAs, and ribozymes that inhibit transcription or translation of the polynucleotide encoding the abl tyrosine kinase.

In preferred embodiments, the inhibitor is formulated in a pharmaceutical composition that enhances the ability of the compound to cross the blood-brain barrier of a mammal. The inhibitor can also be administered directly to the brain of the mammal.

The invention is additionally directed to non-human mammals comprising a transgene encoding an abl tyrosine kinase such that the abl tyrosine kinase is expressed in a neuron of the mammal. Given the knowledge provided herein that abl tyrosine kinases are active in mammalian neurons and are involved in tauopathies, the mammals of these embodiments are useful for studying those tauopathies and in screening potential treatments for those tauopathies. These mammals can be produced without undue experimentation.

The abl tyrosine kinase in these mammals can be abl1 or abl2, or any other abl tyrosine kinase later discovered. Additionally, the transgene encoding the abl tyrosine kinase can be from any mammal, or can be a chimera from more than one mammal, or can comprise non-naturally occurring nucleotides. The transgene can also encode non-naturally occurring amino acids, to study the effect of such substitutions. In preferred embodiments, however, the mammal expresses at least one naturally occurring abl tyrosine kinase, most preferably a human abl tyrosine kinase. In additional preferred embodiments, the mammal expresses both human abl1 and human abl2 in the neuron of the mammal.

The expression of the abl tyrosine kinase in these mammals can be inducible or constitutive, depending on goals of the studies employing the mammals. Preferably, the expression of the abl tyrosine kinase is limited to neurons of the mammal.

The mammal of these embodiments can be of any species, but is preferably an experimental animal such as a dog, cat, guinea pig, rat, or preferably a mouse.

The invention is additionally directed to methods of evaluating whether a compound inhibits development of a tauopathy. The methods comprise combining the compound with an abl tyrosine kinase and determining whether the compound inhibits the abl tyrosine kinase. In these embodiments, a compound that inhibits the abl tyrosine kinase inhibits development of the tauopathy. Here, abl tyrosine kinase can be abl1 or abl2. Preferably, the tyrosine kinase is a human abl tyrosine kinase, most preferably either human abl1 or human abl2.

The tauopathy here can be any tauopathy involving tyrosine phosphorylation, for example frontotemportal dementia, progressive supernuclear palsy, Pick's disease, corticobasal degeneration, Parkinson's disease, or Lewy body dementia. Preferably, the tauopathy is Alzheimer's disease.

These methods can also employ the non-human mammals described above, where the abl tyrosine kinase to be tested for inhibition is in the non-human mammal.

Preferred embodiments of the invention are described in the following Examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### EXAMPLE 1

### Phosphorylation of Tau by Abl and its Relationship to Alzheimer's Disease and Other Tauopathies

The discovery of active cell division mechanisms in neurons of patients with tauopathies such as Alzheimer's disease is almost 10 years old, and numerous papers have established that some aspects of cell division are indeed turned on in neurons that undergo degeneration in this disease. This finding was originally very controversial, because it was very well established that differentiated neurons in the adult brain do not undergo cell division. There is evidently a very strong mechanism preventing cell division: tumors never arise from differentiated neurons: neuroblastoma is completely unknown in adults. On the other hand, evidence that cell division mechanisms were activated in neurons of patients with Alzheimer's disease was very strong, and recent reports also suggest activation of the cell cycle in neurons surrounding strokes. This left two compelling questions:
1. What activates the cell cycle in neurons of patients with Alzheimer's disease (and perhaps following stroke)?
2. What are the consequences of cell cycle activation?

One of the most prominent activators of the cell cycle in white blood cells (lymphocytes) is the non-receptor tyrosine kinase, c-abl (also called abl-1). For many years it has been clear that abnormalities in abl are associated with leukemia, a cancer which occurs when lymphocyte cell division is not normally controlled. There are three different ways in which c-abl abnormalities can cause leukemia, by chromosomal rearrangements producing an abnormal c-abl-containing gene, by mutation of the c-abl gene itself, and by infection with a virus carrying a gene similar to the cellular c-abl gene. All three of these abnormalities result in the production of an abl protein that is more active than the cellular counterpart. It is by increasing the activity of the abl kinase that these abnormalities cause uncontrolled cell division. Inhibiting the abl kinase activity with compounds such as Gleevec is a very effective treatment for many cases of leukemia: when this treatment fails, it is almost always because the abl kinase activity becomes resistant to inhibition.

In non-cancer cells, abl kinase activity is tightly regulated, but it can be activated by damage to DNA (from environmental toxins or radiation) or by oxidative stress. There have been a few reports that toxins or radiation induced activation of abl in neurons, but these have been narrowly focused studies unrelated to the mechanism of Alzheimer's disease.

Exploring a potential link between abl and Alzheimer's based on the rationale above, we established that tau and abl are co-localized in neurofibrillary tangles in Alzheimer's disease (Conrad, 2003).

We further evaluated the role of abl in tau phosphorylation in Alzheimer's disease. Derkinderen et al. (2005) have also studied the interaction of c-abl and tau.

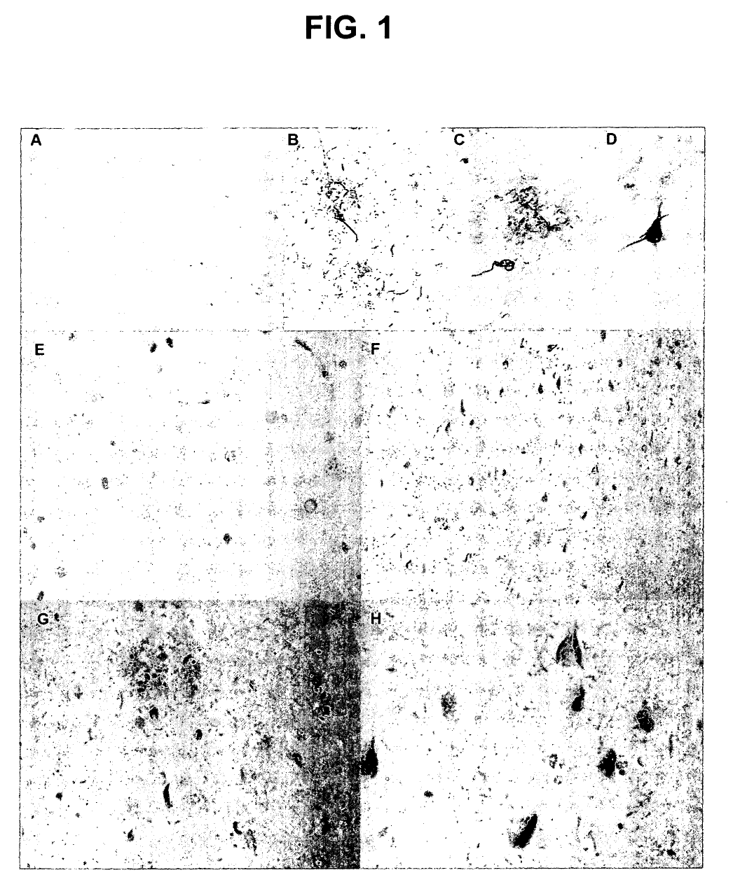

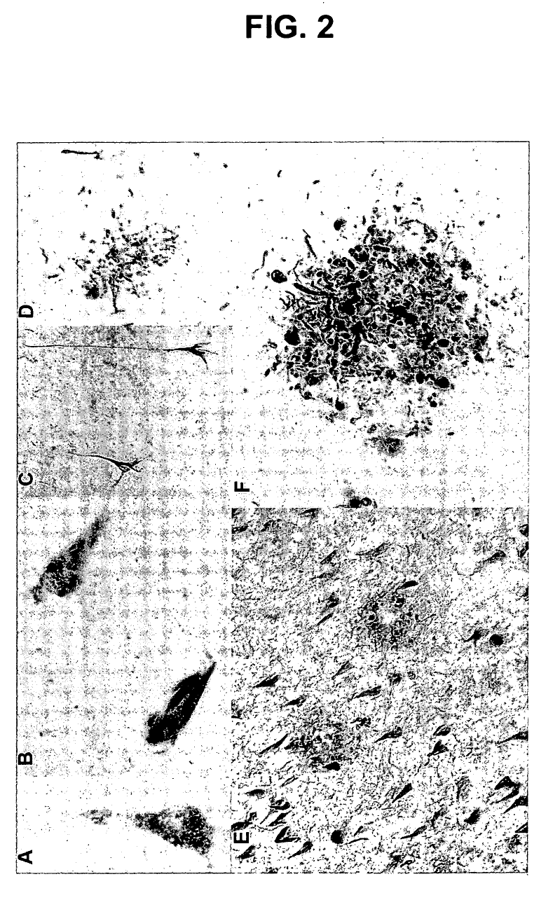

Alzheimer's disease brain sections were stained with anti-abl antibodies. Tangles stained positively for abl (FIG. 1). Additionally, antibodies that recognize only tau phosphorylated at tyr18 or tyr29 but not unphosphorylated tau bound to sections of Alzheimer's patients but not sections of normal adult human brain (FIG. 2). Panels A-D are from a patient with an early case of Alzheimer's disease. Panel A shows a neuron in the brain of this patient. The neuron stains very lightly because it does not have a tangle. Panel B shows two cells, one with a tangle (bottom left) and one with an apparent early tangle (upper right). Panel C shows that cells with tangles stain throughout the cell and the cell processes are clearly visible. Panel D shows the neuronal processes that surround amyloid deposits (the processes in plaques) also stain for tyrosine phosphorylated tau, even in this early Alzheimer's case. Panel E shows the tyrosine phosphorylation in a more advanced Alzheimer's case. Staining is abundant. Both plaques and tangle staining are clearly visible. Panel F is from an advanced Alzheimer's case. This section shows intense staining of the neuronal processes in plaques.

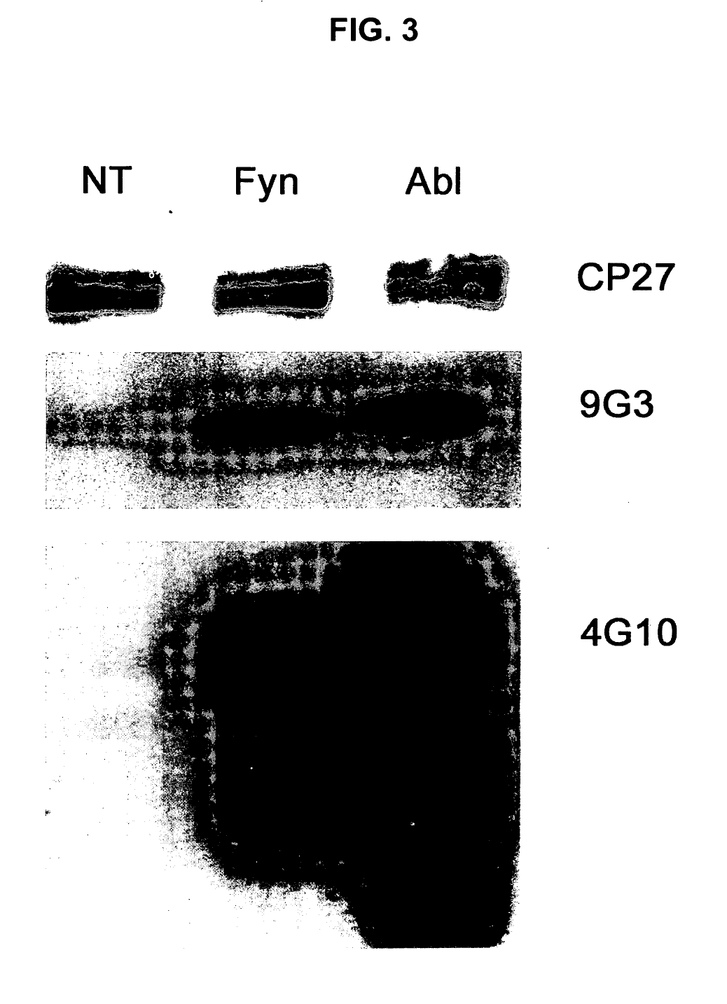

Phosphorylation of tau in cells was then evaluated. Chinese hamster ovary (CHO) cells that expressed transgenic tau were transfected with vectors expressing fyn or abl. Protein from the cells were then subjected to SDS-PAGE then western blotting with antibodies that recognized phosphorylated or unphosphorylated tau. The results are shown in FIG. 3. Abl phosphorylated tau to a much greater extent than fyn.

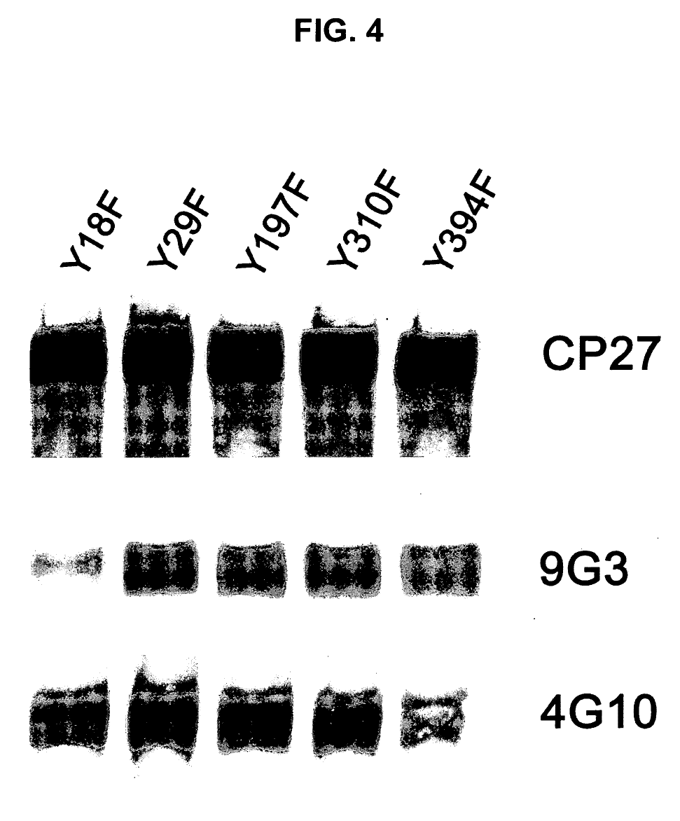

FIG. 4 shows the result of an experiment establishing that tyr394 of tau is phosphorylated. Five mutant tau proteins were synthesized. Each mutant had a different tyr (Y) to phenylalanine (F) mutation. Y18F is mutated at tyr18, etc. Phenylalanine is very similar in structure to tyrosine, but cannot be phosphorylated. The mutant proteins were transfected into cells with abl then lysed and subjected to SDS-PAGE and western blot, as described with FIG. 3. Abl phosphorylated tyrosine 394 best, and also appeared to phosphorylate tyr18 and tyr197, by visual observation. This experiment does not rule out phosphorylation of tyr29 or tyr310 also. Indeed, phosphorylation of tyr310 would be expected by abl because of the amino acid sequence around that residue is vary similar to tyr394.

### EXAMPLE 2

### Further Studies of the Phosphorylation of Tau by Abl

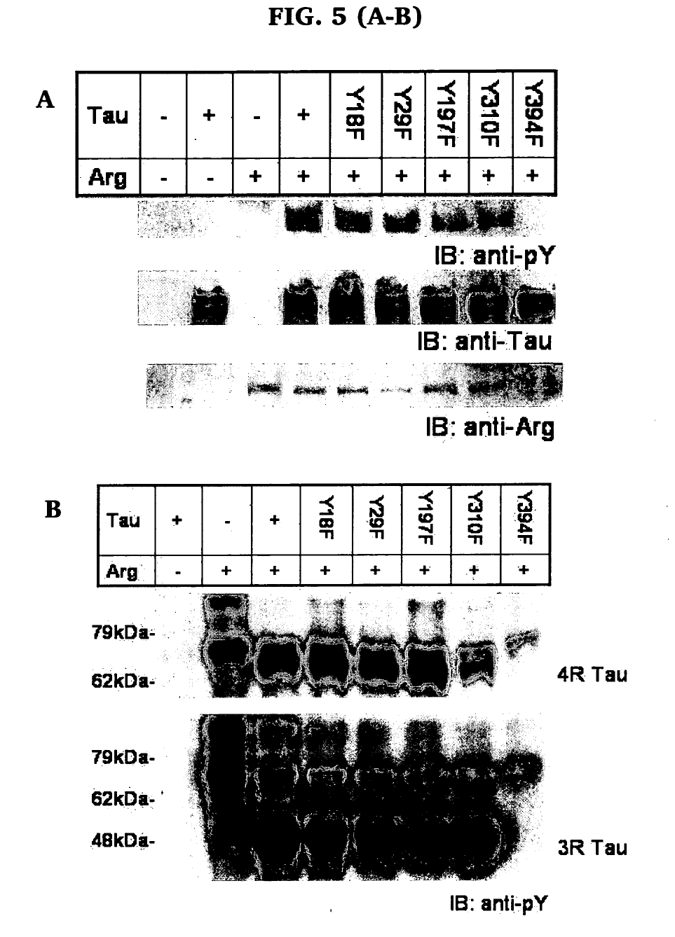

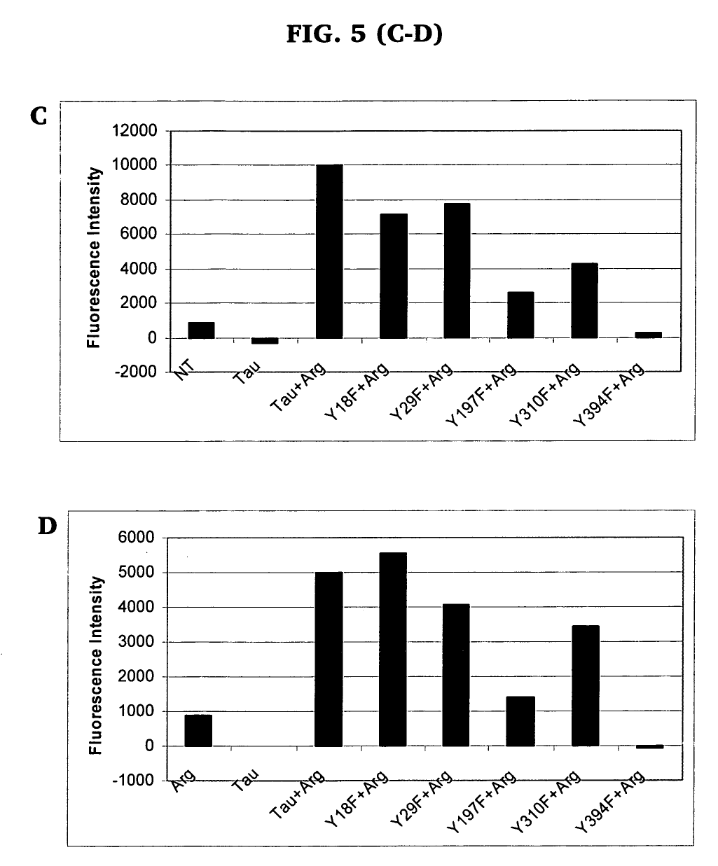

Western blots and ELISA were utilized to evaluate abl2 (Arg) phosphorylation of tau at tyrosine-394 (Y394) and tyrosine 310 (Y310). Cells expressing the Y-to-F mutants described in Example 1 were co-transfected with abl2 and lysates were subjected to western blots and ELISA. FIG. 5A shows the results of staining those lysates with anti-phosphotyrosine (anti-pY), anti-Tau, and anti-abl2 (anti-Arg). Abl2 strongly phosphorylates Y394, with lower levels of phosphorylation at Y197 and Y310. When long (4R) and short (3R) isoforms of the mutant tau SEQ ID Nos 2 and 1, respectively) were cotransfected with abl2 (arg), Y394 was the predominant site of abl2 phosphorylation (FIG. 5B). Sandwich ELISA confirms western blotting results indicating Y394 as the major phosphorylation site for both the longest (FIG. 5C) and shortest (FIG. 5D) isoforms of tau.

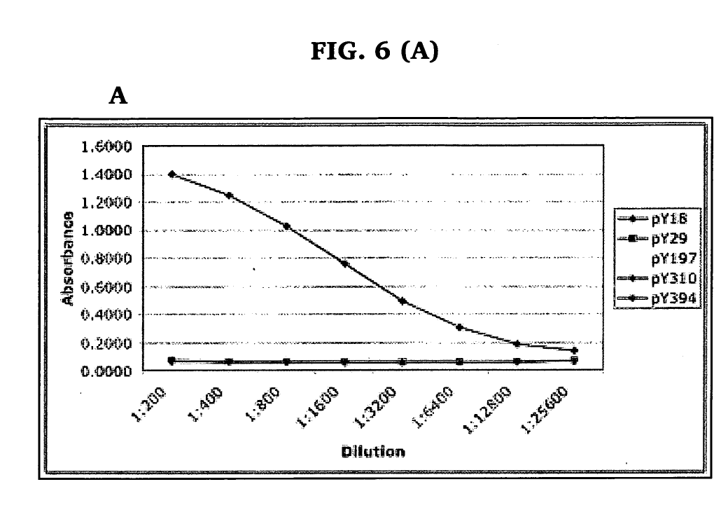

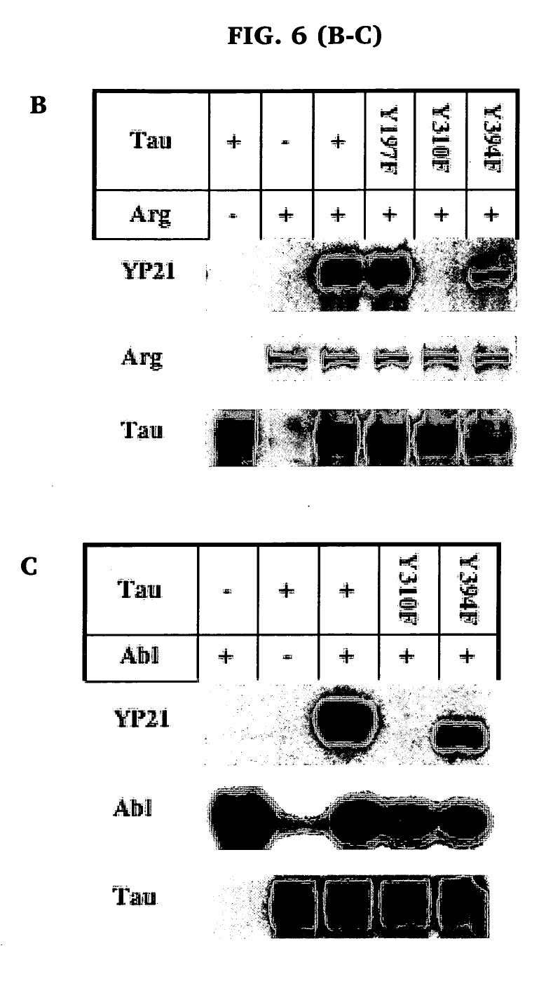

A monoclonal antibody (YP21) was developed that specifically recognizes tau phosphorylated at Y310. The specificity of that antibody was established using ELISA to measure affinity of the antibody for various phospho-tyrosine containing tau peptides (FIG. 6A). This antibody was used in western blots with cell lysates from cotransfection of tau with both abl2 (arg) (FIG. 6B) and abl1 (FIG. 6C), demonstrating phosphorylation of tau at Y310, and a complete loss of YP21 immunoreactivity when Y310 is mutated.

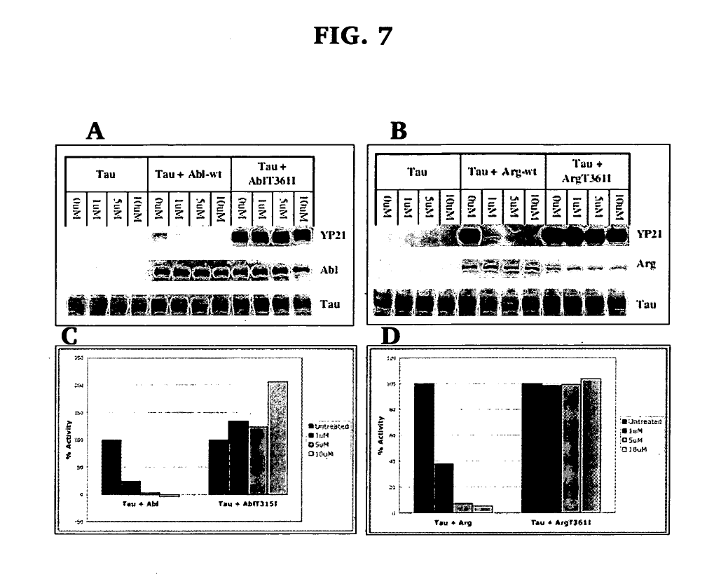

To evaluate the ability of abl1 and abl2 to independently phosphorylate tau, T3611 forms of abl1 and abl2 that are resistant to Gleevec (imatinib mesylate) (Young et al., 2006) were made and transfected into cells with abl1 or abl2, and lysates were evaluated by western blot (FIG. 7A [abl1] and 7B [abl2] and ELISA (FIG. 7C [abl1] and 7D [abl2]). In the presence of Gleevec, both forms could phosphorylate tau. This establishes that abl2 activity does not result in tau phosphorylation by activating abl, or vice versa. Abl1 or abl2 transfected into the cell is what directly phosphorylates tau, since tyrosine phosphorylation of tau is retained in the presence of drug resistant forms of each Abl family kinase, regardless of Gleevec treatment.

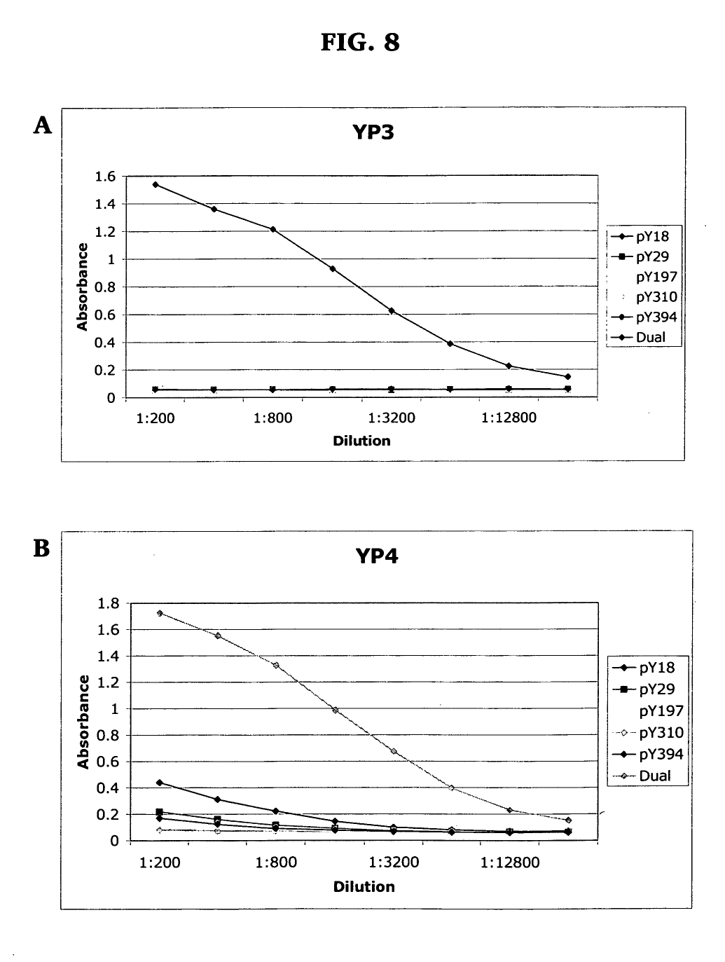

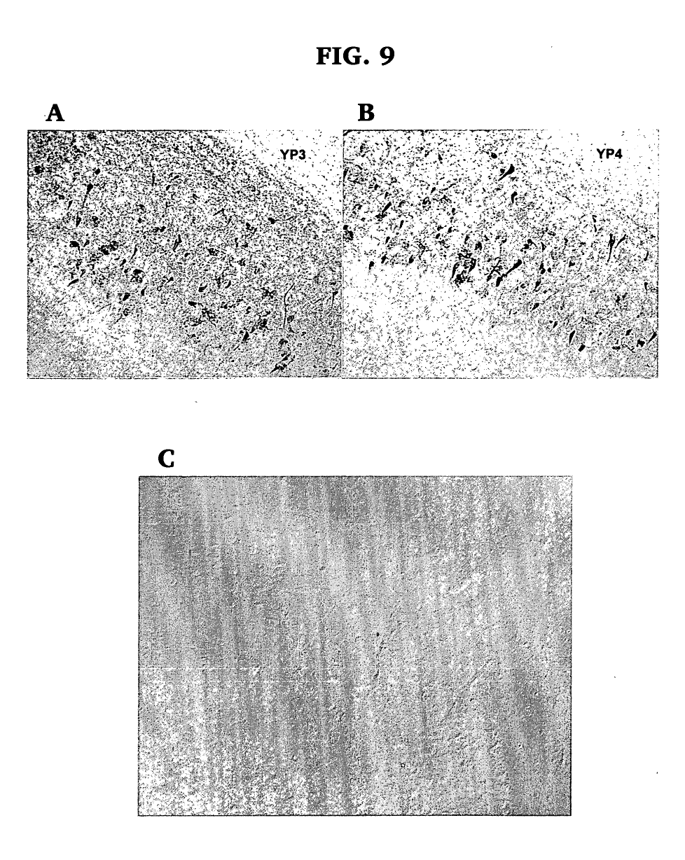

Serine 396 of tau is always phosphorylated in Alzheimer's disease (Uboga and Price, 2000; Weaver et al., 2000). Thus abl1 or abl2 activity should produce a dual phosphorylated site, phosphotyrosine 394/phosphoserine 396. Two monoclonal antibodies against that site, designated YP3 and YP4, were developed (FIG. 8). These antibodies do not see tau when only tyrosine 394 is phosphorylated, nor do they see tau when only 396 is phosphorylated, but are specific for the dual phosphorylation (FIG. 8). Sites immunoreactive to these antibodies are present in large amounts in the Alzheimer brain, in association with plaques, tangles and abnormal neuritis (FIGS. 9A and 9B). No reactivity of these antibodies with normal brain is found (FIG. 9C, shown only for YP3, but normal tissue is also negative with YP4).

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

SEQ ID NO:s

```
SEQ ID NO:1 Human tau, isoform 4 (tau352) amino acid sequence. From
GenBank NP058525 tyrosines are bold - corresponding to 18, 29, 197,
310, 394 of the long isoform 4 (SEQ ID NO:2)
    1 maeprqefev medhagtygl gdrkdqggyt mhqdqegdtd aglkaeeagi gdtpsledea
  61 aghvtqarmv skskdgtgsd dkkakgadgk tkiatprgaa ppgqkgqana tripaktppa
121 pktppssgep pksgdrsgys spgspgtpgs rsrtpslptp ptrepkkvav vrtppkspss
181 aksrlqtapv pmpdlknvks kigstenlkh qpgggkvqiv ykpvdlskvt skcgslgnih
241 hkpgggqvev ksekldfkdr vqskigsldn ithvpgggnk kiethkltfr enakaktdhg
301 aeivykspvv sgdtsprhls nvsstgsidm vdspqlatla devsaslakq gl
SEQ ID NO:2 Human tau, isoform 2 (tau441) amino acid sequence. From
GenBank NP005901 tyrosines are bold - 18, 29, 197, 310, 394
    1 maeprqefev medhagtygl gdrkdqggyt mhqdqegdtd aglkesplqt ptedgseepg
  61 setsdakstp taedvtaplv degapgkqaa aqphteipeg ttaeeagigd tpsledeaag
121 hvtqarmvsk skdgtgsddk kakgadgktk iatprgaapp gqkgqanatr ipaktppapk
181 tppssgeppk sgdrsgyssp gspgtpgsrs rtpslptppt repkkvavvr tppkspssak
241 srlqtapvpm pdlknvkski gstenlkhqp gggkvqiink kldlsnvqsk cgskdnikhv
301 pgggsvqivy kpvdlskvts kcgslgnihh kpgggqvevk sekldfkdrv qskigsldni
361 thvpgggnkk iethkltfre nakaktdhga eivykspvvs gdtsprhlsn vsstgsidmv
421 dspqlatlad evsaslakqg l
SEQ ID NO:3 abl1 amino acid sequence. From GenBank P00519
      1 mleiclklvg ckskkglsss sscyleealq rpvasdfepq glseaarwns kenllagpse
    61 ndpnlfvaly dfvasgdntl sitkgeklrv lgynhngewc eaqtkngqgw vpsnyitpvn
  121 slekhswyhg pvsrnaaeyl lssgingsfl vresesspgq rsislryegr vyhyrintas
  181 dgklyvsses rfntlaelvh hhstvadgli ttlhypapkr nkptvygvsp nydkwemert
  241 ditmkhklgg gqygevyegv wkkysltvav ktlkedtmev eeflkeaavm keikhpnlvq
  301 llgvctrepp fyiitefmty gnlldylrec nrqevnavvl lymatqissa meylekknfi
  361 hrdlaarncl vgenhlvkva dfglsrlmtg dtytahagak fpikwtapes laynkfsiks
  421 dvwafgvllw eiatygmspy pgidlsqvye llekdyrmer pegcpekvye lmracwqwnp
  481 sdrpsfaeih qafetmfqes sisdevekel gkqgvrgavs tllqapelpt ktrtsrraae
  541 hrdttdvpem phskgqgesd pldhepavsp llprkergpp egginederi lpkdkktnlf
  601 salikkkkkt aptppkrsss fremdgqper rgageeegrd isngalaftp ldtadpaksp
  661 kpsngagvpn galresggsg frsphlwkks stltssrlat geeegggsss krflrscsas
  721 cvphgakdte wrsvtlprdl qstgrqfdss tfgghksekp alprkragen rsdqvtrgtv
  781 tppprlvkkn eeaadevfkd imesspgssp pnltpkplrr qvtvapasgl phkeeaekgs
  841 algtpaaaep vtptskagsg apggtskgpa eesrvrrhkh ssespgrdkg klsrlkpapp
  901 pppaasagka ggkpsqspsq eaageavlga ktkatslvda vnsdaakpsq pgeglkkpvl
  961 patpkpqsak psgtpispap vpstlpsass alagdqpsst afiplistrv slrktrqppe
1021 riasgaitkg vvldstealc laisrnseqm ashsavleag knlytfcvsy vdsiqqmrnk
1081 fafreainkl ennirelqic patagsgpaa tqdfskllss vkeisdivqr
SEQ ID NO:3 abl2 amino acid sequence. From GenBank P42684
      1 mgqqvgrvge apglqqpqpr girgssaarp sgrrrdpagr ttetgfnift qhdhfascve
    61 dgfegdktgg sspealhrpy gcdvepqaln eairwssken llgatesdpn lfvalydfva
  121 sgdntlsitk geklrvlgyn qngewsevrs kngqgwvpsn yitpvnslek hswyhgpvsr
  181 saaeyllssl ingsflvres esspgqlsis lryegrvyhy rinttadgkv yvtaesrfst
  241 laelvhhhst vadglvttlh ypapkcnkpt vygvspihdk wemertditm khklgggqyg
  301 evyvgvwkky sltvavktlk edtmeveefl keaavmkeik hpnlvqllgv ctleppfyiv
  361 teympygnll dylrecnree vtavvllyma tqissameyl ekknfihrdl aarnclvgen
  421 hvvkvadfgl srlmtgdtyt ahagakfpik wtapeslayn tfsiksdvwa fgvllweiat
  481 ygmspypgid lsqvydllek gyrmeqpegc ppkvyelmra cwkwspadrp sfaethqafe
  541 tmfhdssise evaeelgraa ssssvvpylp rlpilpsktr tlkkqvenke niegaqdate
  601 nsasslapgf irgaqassgs palprkqrdk spsslledak etcftrdrkg gffssfmkkr
  661 naptppkrss sfremenqph kkyeltgnfs svaslqhadg fsftpaqqea nlvppkcygg
  721 sfaqrnlcnd dggggggsgt agggwsgitg fftprlikkt lglragkpta sddtskpfpr
  781 snstssmssg lpeqdrmamt lprncqrskl qlertvstss qpeenvdran dmlpkksees
  841 aapsrerpka kllprgatal plrtpsgdla itekdppgvg vagvaaapkg keknggarlg
  901 magvpedgeq pgwpspakaa pvlptthnhk vpvlisptlk htpadvqlig tdsqgnkfld
  961 lsehqvtssg dkdrprrvkp kcapppppvm rllqhpsics dpteeptalt agqstsetqe
1021 ggkkaalgav pisgkagrpv mpppqvplpt ssispakman gtagtkvalr ktkqaaekis
1081 adkiskeall ecadllssal tepvpnsqlv dtghqlldyc sgyvdcipqt rnkfafreav
1141 sklelslqel qvssaaagvp gtnpvlnnll scvqeisdvv qr
```

1. A method of diagnosing a tauopathy in a subject, the method comprising determining whether the subject has phosphorylation of tau at tyr394 or tyr310, wherein phosphorylation of tau at tyr394 or tyr310 in the subject indicates that the subject has a tauopathy.

2. The method of claim 1, wherein the method comprises determining whether the subject has phosphorylation of tau at tyr394.

3. The method of claim 1, wherein the method comprises determining whether the subject has phosphorylation of tau at tyr310.

4. The method of claim 1, further comprising determining whether the subject has phosphorylation of tau at a second amino acid residue.

5. The method of claim 4, wherein the method comprises determining whether the subject has phosphorylation of tau at tyr394 and ser396.

6. The method of claim 5, wherein phosphorylation of tau at tyr394 and ser396 is determined using an antibody that binds specifically to tau phosphorylated at tyr394 and ser396.

7. The method of claim 6, wherein the antibody is antibody YP3 or YP4.

8. The method of claim 4, wherein the second amino acid residue is a tyrosine.

9. The method of claim 8, wherein the method comprises determining whether the subject has phosphorylation of tau at tyr394 and tyr310.

10. The method of claim 1, wherein the tauopathy is frontotemportal dementia, progressive supernuclear palsy, Pick's disease, corticobasal degeneration, Parkinson's disease, Lewy body dementia, or Alzheimer's disease.

11. The method of claim 1, wherein the tauopathy is Alzheimer's disease.

12. The method of claim 1, where tyrosine phosphorylation of tau at tyr394 or tyr310 is determined in a bodily fluid of the subject.

13. The method of claim 12, wherein the bodily fluid is peripheral blood or cerebrospinal fluid.

14. The method of claim 1, wherein the tyrosine phosphorylation of tau is determined using an antibody specific for tau phosphorylated at tyr394 or tyr310.

15. A method of predicting whether a subject will develop a tauopathy, the method comprising determining whether the subject has tau phosphorylated at tyr394 or tyr310, wherein the presence in the subject of tau phosphorylated at tyr394 or tyr310 indicates the subject will develop the tauopathy.

16-28. (canceled)

29. An antibody preparation that specifically binds to tau phosphorylated at tyr394 and/or tyr310.

30-33. (canceled)

34. The antibody preparation of claim 29, wherein the preparation specifically binds to tau phosphorylated at tyr394 and ser396.

35. The antibody preparation of claim 34, wherein the preparation comprises YP3 or YP4 antibodies.

36. (canceled)

37. A method of inhibiting tau phosphorylation in a cell, the method comprising combining the cell with an inhibitor of an abl tyrosine kinase in a manner sufficient to inhibit tau phosphorylation in the cell.

38-54. (canceled)

55. A method of treating a subject having a tauopathy, the method comprising administering an inhibitor of an abl tyrosine kinase to the subject in a manner sufficient to inhibit tau tyrosine phosphorylation in a neuron in the subject.

56-57. (canceled)

58. The method of claim 55, wherein the tauopathy is frontotemportal dementia, progressive supernuclear palsy, Pick's disease, corticobasal degeneration, Parkinson's disease, Lewy body dementia, or Alzheimer's disease.

59-62. (canceled)

63. The method of claim 55, wherein the inhibitor is ST1571, CGP57148, AG1112, AP23464 or PP1.

64-68. (canceled)

69. A method of treating a subject at risk for a tauopathy, the method comprising administering an inhibitor of an abl tyrosine kinase to the subject in a manner sufficient to inhibit tau tyrosine phosphorylation in a neuron in the subject.

70-82. (canceled)

83. A non-human mammal comprising a transgene encoding an abl tyrosine kinase such that the abl tyrosine kinase is expressed in a neuron of the mammal.

84-89. (canceled)

90. A method of evaluating whether a compound inhibits development of a tauopathy, the method comprising combining the compound with an abl tyrosine kinase and determining whether the compound inhibits the abl tyrosine kinase, wherein a compound that inhibits the abl tyrosine kinase inhibits development of the tauopathy.

91-98. (canceled)
